(19) United States
(12) Patent Application Publication
(10) Pub. No.: US 2022/0322769 A1
(43) Pub. Date: Oct. 13, 2022

(54) Compression Garment

(71) Applicant: Misti King, Tyler, TX (US)

(72) Inventor: Misti King, Tyler, TX (US)

(21) Appl. No.: 17/716,043

(22) Filed: Apr. 8, 2022

Related U.S. Application Data
(60) Provisional application No. 63/172,918, filed on Apr. 9, 2021.

(51) Int. Cl. A41D 13/12; A61F 13/08; A41D 1/089; A41D 1/06; A41D 27/20

(52) U.S. Cl. CPC A41D 13/1254; A61F 13/08; A41D 1/089; A41D 1/065; A41D 27/20; A41D 2400/44; A41D 2300/32; A41D 2300/326

(57) ABSTRACT

The present invention relates to a compression garment designed to be worn by women, pregnant or post-partum, for body-shaping and/or after an injury or surgery. The compression garment comprises a plurality of hooks or clips sewn to the garment to which a plurality of straps are attached. The straps are grasped by a user to easily pull on and off the garment. The straps can be stored in a pocket disposed in the garment for a seamless appearance. The compression garment has a removable crotch region to be removed when a user uses a restroom, thereby eliminating removal of the entire compression garment.

### CROSS-REFERENCE TO RELATED APPLICATION

The present application claims priority to, and the benefit of, U.S. Provisional Application No. 63/172,918, which was filed on Apr. 9, 2021 and is incorporated herein by reference in its entirety.

### FIELD OF THE INVENTION

The present invention relates generally to compression garments. More specifically, the present invention relates to a compression garment with removably-secured straps. The compression garment comprises a plurality of hooks to which a plurality of straps are detachably-fastened to allow a user to grasp the straps to easily pull on and off the compression garment. The compression garment also comprises a pocket to store said straps, and a removable crotch portion configured to be removed when using a restroom without requiring a user to remove the complete compression garment. Accordingly, the present disclosure makes specific reference thereto. Nonetheless, it is to be appreciated that aspects of the present invention are also equally applicable to other like applications, devices and methods of manufacture.

### BACKGROUND OF THE INVENTION

By way of background, compression garments are worn for reducing swelling after surgery, after giving birth and also for everyday body-shaping. Especially for females, compression garments can be used both during pregnancy and post-pregnancy. During pregnancy, some women suffer from vulval varicosities and pelvic instability, and compression garments can help in both reducing the pain and in treatment of these conditions.

Additionally, women who are post-partum may have difficulty regaining their pre-pregnancy figure and function. Specifically, the skin over the abdomen, along with the abdominal muscles, stretches and internal organs may displace. Compression garments can help women attain their pre-pregnancy shape.

Also, many users face problems with abdominal fat protruding over and around their clothing, especially with lower-body garments that cinch in at the waist. Compression garments can restrain, smooth and conceal undesirable bulges of abdominal fat and provide a desirable shape for the user.

The compression garments have a compressive nature and as a result of their compressive properties, compression garments are typically very difficult and cumbersome to pull on and remove. This problem is aggravated when compression garments are pulled on over an injury or surgical incision area. Further, users may need to ask for help when pulling on or removing a compression garment, which may be embarrassing and inconvenient for both parties involved.

Compression garments are also commonly used after a liposuction procedure. The compression garments facilitate drainage of anesthetic solutions and prevent accumulation of fluid within the tunnels in the subcutaneous fat created by the suction cannula. In addition, the compression garments can hold bulky absorbent dressings in place, all of which helps to minimize the discomfort associated with the copious drainage that may occur after surgery.

Further, users may face difficulties when using a restroom while wearing compression garments, especially those made for the lower body. Additionally, when the compression garment is worn for an entire day, such as during work, it becomes extremely difficult for users to remove the compression garment. This can be troublesome for post-surgical and pregnant users. An easier and more accessible way for a user to use the restroom without pulling on and off his or her own compression garment is therefore desired.

Currently aids for pulling on and off compression garments such as rubber gloves, lotions, etc., provide some assistance. However, such aids have to be purchased separately, are relatively expensive and may occupy additional space while travelling. An easier way for a user to pull on and off his or her own compression garment is therefore desired.

Therefore, there exists a long felt need in the art for an improved compression garment that can be easily pulled on and off by a user. There is also a long felt need in the art for an improved compression garment that can be easily worn over an area with a recent injury, like a wound or surgical incision site. Additionally, there is a long felt need in the art for an improved compression garment that can be pulled on and off without help from others. Further, there is a long felt need in the art for an improved compression garment that can be used during pregnancy, post-pregnancy and rehab after surgery. Moreover, there is a long felt need in the art for an improved compression garment that allows users to use a restroom without removing the compression garment. Also, there is a long felt need in the art for an improved compression garment that is cost-effective and easy to carry. Finally, there is a long felt need in the art for an improved compression garment that provides uniform compression, and can be worn and removed easily without help from others.

The subject matter disclosed and claimed herein, in one embodiment thereof, comprises a compression garment. The compression garment is designed for users with recent injuries or surgeries, during pregnancy and post-partum. The garment is also useful for daily body-shaping. The compression garment comprises a stretchable and breathable material, a front surface, a rear surface, and a removable crotch portion for covering the crotch area of a user. A plurality of hooks are disposed on the front surface and the rear surface, to which a plurality of straps are engaged for allowing a user to grip the straps for easily pulling up and down the compression garment. The compression garment comprises a pocket to store said straps, and the removable crotch portion allows a user to use a restroom without removing the entire compression garment.

In this manner, the improved compression garment of the present invention accomplishes all of the forgoing objectives and provides a relatively easy and convenient garment that can be worn over injured areas easily without help from others. The improved compression garment features several straps and handles that allow a user to easily pull the garment on and off of a user's body. The improved compression garment can be used by pregnant women, post-partum women, post-surgery users, etc.

### SUMMARY OF THE INVENTION

The following presents a simplified summary in order to provide a basic understanding of some aspects of the disclosed innovation. This summary is not an extensive overview, and it is not intended to identify key/critical elements or to delineate the scope thereof. Its sole purpose is to present some general concepts in a simplified form as a prelude to the more detailed description that is presented later.

The subject matter disclosed and claimed herein, in one embodiment thereof, comprises a compression garment. The compression garment is designed for users with recent injuries, during pregnancy and post-partum. The compression garment comprises a one-piece garment made up of a stretchable and breathable material, a front surface, a rear surface, a removable crotch portion for covering the crotch area of a user, a plurality of hooks disposed on the front surface and the rear surface, and a plurality of straps wherein each strap engages a pair of hooks, allowing a user to grasp the straps to easily pull on and off the compression garment. The compression garment has a pocket to store said straps, and the removable crotch portion is removed to allow a user to use a restroom without removing the entire compression garment.

In yet another embodiment of the present invention, the compression garment is a unitary structure made up of single compressive material. The crotch portion is attached through hook and loop or Velcro® fasteners. Other suitable fasteners known to those of ordinary skill in the art may also be used.

In another embodiment of the present invention, a compression garment for a user's lower body is disclosed. The compression garment comprises a one-piece garment of a breathable and elastic material, adapted to be worn around the lower body, the one-piece garment has a left leg opening and a right leg opening, a waste removal removable crotch portion, a plurality of hooks disposed on the garment which are designed to receive straps, wherein the straps have loops to attach to said hooks, and a pocket to store said straps when they are not engaged with said hooks. Each strap has loops at the ends that are designed to fasten to a pair of hooks in either a horizontal or vertical direction.

In yet another embodiment of the present invention, the straps, when attached to the hooks, are grasped by a user allowing the user to easily pull up or down the compression garment without help from others.

In a further embodiment of the present invention, a method for donning a compression garment is described. The method includes providing the compression garment, attaching a plurality of straps to a plurality of hooks that are sewn to the compression garment, wherein each strap is sewn to a pair of hooks, inserting limbs into the leg openings of the compression garment, pulling the compression garment by grasping the straps with a firm and secure grip, and when the garment is worn over the body removing the straps and storing the straps in a pocket disposed in the garment.

In yet another embodiment of the present invention, the method further includes removing a crotch portion when a user uses a restroom, and re-attaching the crotch portion afterwards. In yet another embodiment of the present invention, the compressive material is durable, flexible and breathable, and made up of a mixture of nylon and spandex. In yet another embodiment of the present invention, the hooks are spaced such that the loops of the straps are fastened to the hooks for a secure attachment.

Advantageously, the compressive garment allows people with a recent injury, such as a wound or surgical incision site, to easily pull on and off the compressive garment. The garment features attachable straps and handles located at multiple areas, that can be hidden within a pocket or covered with compression material, and are used for pulling the garment on and off over a user's body.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and are intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1 illustrates a front perspective view of one embodiment of the improved compression garment of the present invention in accordance with the disclosed architecture;

FIG. 2 illustrates a rear perspective view of one embodiment of the improved compression garment of the present invention in accordance with the disclosed architecture;

FIG. 3 illustrates a perspective view showing the hooks on the improved compression garment for attaching the removably secured straps in accordance with the disclosed architecture;

FIG. 4 illustrates a perspective view showing how the removable crotch area is removed from the compression garment in accordance with the disclosed architecture; and

FIG. 5 illustrates a flow diagram showing the steps of pulling up the compression garment over the body in accordance with the disclosed architecture.

### DETAILED DESCRIPTION OF THE PRESENT INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof. Various embodiments are discussed hereinafter. It should be noted that the figures are described only to facilitate the description of the embodiments. They are not intended as an exhaustive description of the invention, and do not limit the scope of the invention. Additionally, an illustrated embodiment need not have all the aspects or advantages shown. Thus, in other embodiments, any of the features described herein from different embodiments may be combined.

As stated supra, there is a long felt need in the art for an improved compression garment that can be easily pulled on and off of a user. There is also a long felt need in the art for an improved compression garment that can be easily worn over an area with a recent injury, such as a wound or a surgical incision site. Additionally, there is a long felt need in the art for an improved compression garment that can be pulled on and off without help from others. Further, there is a long felt need in the art for an improved compression garment that can be used during pregnancy, postpartum and rehab after surgery. Moreover, there is a long felt need in the art for an improved compression garment that allows users to be able to use a restroom without removing the compression garment. Also, there is a long felt need in the art for an improved compression garment that is cost-effective and easy to carry. Finally, there is a long felt need in the art for an improved compression garment that provides uniform compression, and can be worn and removed easily without help from others.

The present invention, in one exemplary embodiment, is a compression garment for a user's lower body. The compression garment comprises a one-piece garment of a breathable and elastic material, adapted to be worn around the lower body. The one-piece garment has a left leg opening and a right leg opening, a removable crotch portion, a plurality of hooks disposed on the garment and designed to receive straps, wherein the straps have loops to attach to said hooks and a pocket to store said straps when they are not engaged to said hooks. Each strap has loops at the ends that are designed to fasten to a pair of hooks in either a horizontal or vertical direction.

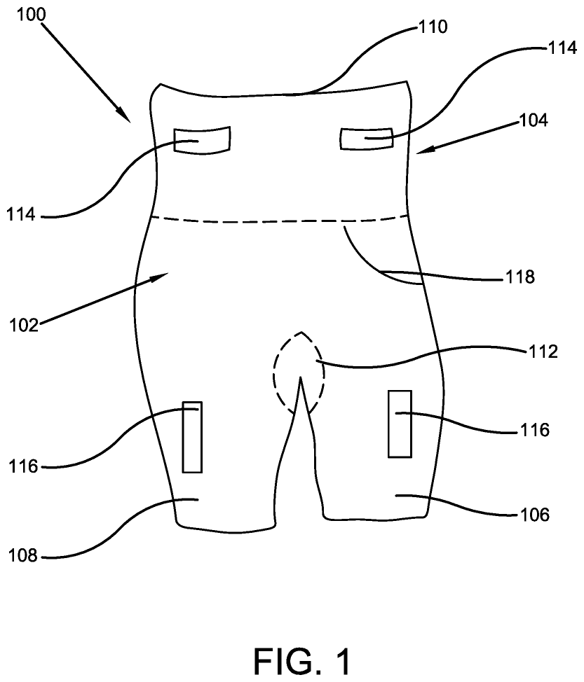

Referring initially to the drawings, FIG. 1 illustrates a front perspective view of one embodiment of the improved compression garment of the present invention. The improved compression garment device 100 is configured to be worn over a user's lower body. The improved compression garment device 100 comprises a front surface 102 configured to adhere to the front body portion of the user, and a rear surface 104 configured to adhere to the rear body portion of the user. The improved compression garment device 100 in the present embodiment is configured in the form of shorts, having a left leg opening 106 and a right leg opening 108 and wherein a material section extends up to the torso of the user with a torso band 110 wrapping around the torso. The improved compression garment device 100 is a one-piece component of a typical compressive material used in conventional compressive garments, or any other suitable compressive material as is known in the art.

The compression garment device 100 comprises a removable crotch area 112 allowing a user easy use of a restroom, without removing the entire compression garment device 100 and soiling their clothes. The removable crotch area 112 is attached to the compression garment device 100 using a fastening mechanism such as a hook and loop fastener or Velcro®, etc., or any other suitable fastening means as is known in the art. Specifically, the removable crotch area 112 can have a hook and loop fastener and the compression garment device 100 can have a hook and loop fastener that are configured to connect with (or mate with) each other to fasten the removable crotch area 112 to the garment device 100 at the crotch opening. The removable crotch area 112 is positioned on the compressive garment device 100 such that it is disposed to cover the crotch region of a user, allowing the user to easily remove the removable crotch area 112 while using a restroom, thereby eliminating removal of the entire compression garment device 100.

The improved compression garment device 100 also comprises a plurality of removably secured straps 114, 116 positioned at various places on the front portion 102. The straps 114, 116 are manufactured of a soft yet durable fabric, and alternatively can be manufactured of nylon or polyester, etc., or any other suitable material as is known in the art. In use, the straps 114, 116 are grasped by a user to maintain a secure grip while the user is pulling on or off the compression garment device 100. The straps 114, 116 provide a user the necessary grip which allow the user to apply the necessary force in which to pull on the garment device 100. In the present embodiment, the front portion 102 comprises horizontal straps 114 positioned near the torso-covering portion, and comprises vertical straps 116 positioned on each leg portion as per a user's needs and/or wants. Once the straps 114, 116 are used for pulling on or off the garment device 100, the straps 114, 166 can be removed and stored for later use, thus providing a seamless look for the garment device 100. For ease in storing the straps 114, 116, the front portion 102 comprises a pocket 118 in which the straps 114, 116 can be stored without fear of losing them. The pocket 118 provides a storage area for the straps 114, 116, while also providing a seamless look to the compression garment device 100. The pocket 118 can be positioned at any suitable position on the device 100.

Once pulled on, the hooked compression garment device 100 easily wraps around a user's body to relieve pain and also to smooth, restrain and conceal undesirable bulges for a more desired body shape. The compression garment device 100 provides desired breathability and moisture vapor transfer. Preferably, the compressive material used for the compression garment device 100 is elasticized and comprises 70-90% nylon yarn with 30-10% spandex. The yarn is preferably nylon, or any other suitable material as is known in the art. Nylon is smooth and provides a silky feel to a user's skin, thereby allowing the compression garment device 100 to be worn over a wound or surgical incision site. Typically, spandex is the compression clothing material that provides the stretch within the garment device 100. However, any other elasticized material can be used as is known in the art. When spandex is blended with nylon, the compression garment device 100 provides a strong and stretchable fabric.

The one-piece compression garment device 100 of the present embodiment can also provide cooler temperatures for the user by retaining less body heat during garment use. The compression garment device 100 provides for reduced tension in the abdominal and leg muscles, along with reduced tension over a surgical wound site.

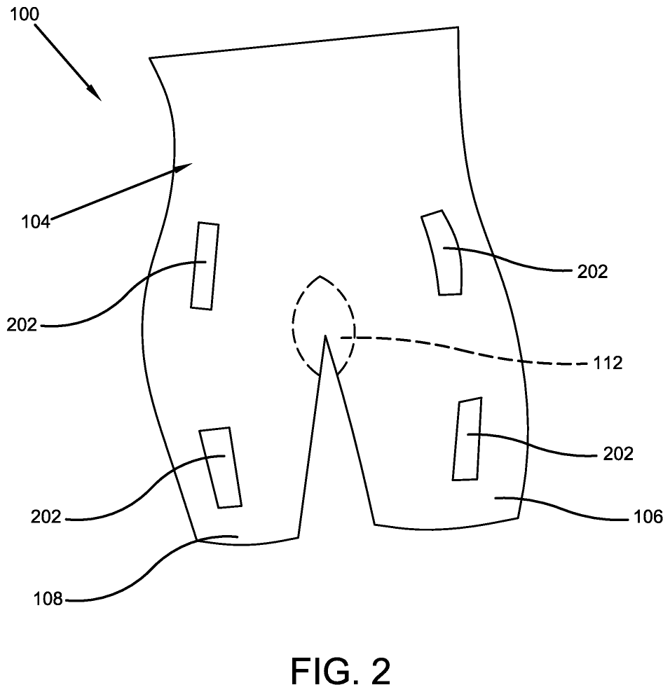

FIG. 2 illustrates a rear perspective view of one embodiment of the improved compression garment of the present invention. The rear surface 104 adheres to the back part of a user's body and conforms to the shape of a user's back. The rear surface 104 comprises a plurality of straps 202 that are similar in size and construction as the straps present on the front surface, as described in FIG. 1. The rear surface 104 also has a removable crotch portion 112, thereby allowing the user to use a restroom without removing the entire compression garment 100. The straps on the front surface and the rear surface allow a user to easily pull on and off the compression garment device 100 without help from others.

The compression garment device 100 is ideal for use in the support and reduction of post-partum loose skin and tissue. Further, the garment device 100 is breathable and allows for stretchability to provide a comfortable garment during installation and use, while also providing skin and muscle shrinkage. The compression garment device 100 comes in a variety of sizes to meet the requirements and/or wants of various types of users, and can have both horizontal and vertical straps or handles.

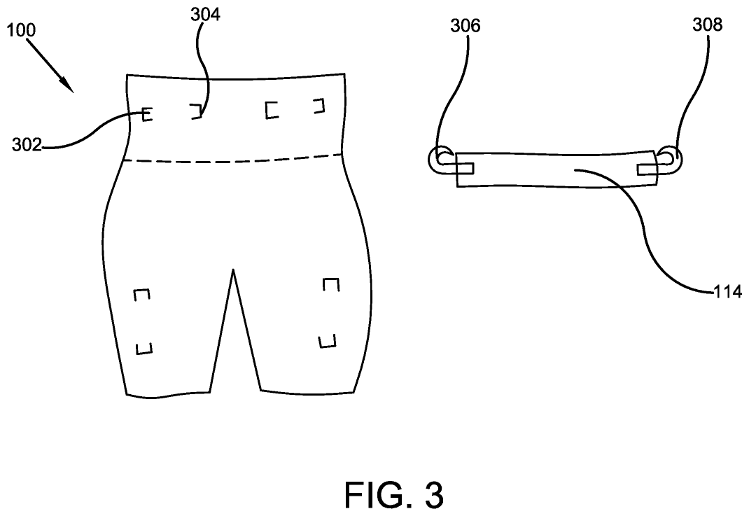

FIG. 3 illustrates a perspective view showing the hooks on the improved compression garment device for attaching the removably-secured straps. In the present embodiment, the details of the attachment are shown for an exemplary strap and mating hooks disposed on the garment device 100. However, it should be appreciated that the same mechanism of attachment can be applied for all the straps, both horizontal and vertical, on the front surface and rear surface of the compression garment device 100, and that the straps can be positioned on any suitable location on the garment device 100 based on the needs and/or wants of a user.

Each strap 114 has a pair of loops 306, 308 at opposing ends. Each loop 306, 308 is designed to be clipped into the hooks 302, 304 disposed on the compression garment device 100. For example, the first loop 306 engages or clips to the first hook 302, and the second loop 308 engages the second hook 304, thereby allowing the strap 114 to be removably-secured to the compression garment device 100. The strap 114 remains secure to the garment device 100, allowing a user to grasp the strap 114 to pull on or off the garment device 100 over his or her body.

Based on the orientation of the pair of hooks 302, 304, the straps 114 can be attached either horizontally or vertically. When the straps 114 are not required, the straps 114 can be removed along with the hooks 302, 304. Alternatively, the hooks 302, 304 are permanently sewn to the compression garment device 100, and may be covered with a thin fabric, thereby giving the compression garment device 100 a seamless appearance.

In an alternative embodiment, the compression garment device 100 can comprise tiny buckles or threaded C-clips. The hooks, buckles or any other fasteners for attaching the straps as known in the art, do not snag the material of the garment device 100 and help in sliding the garment device 100 over a user's body.

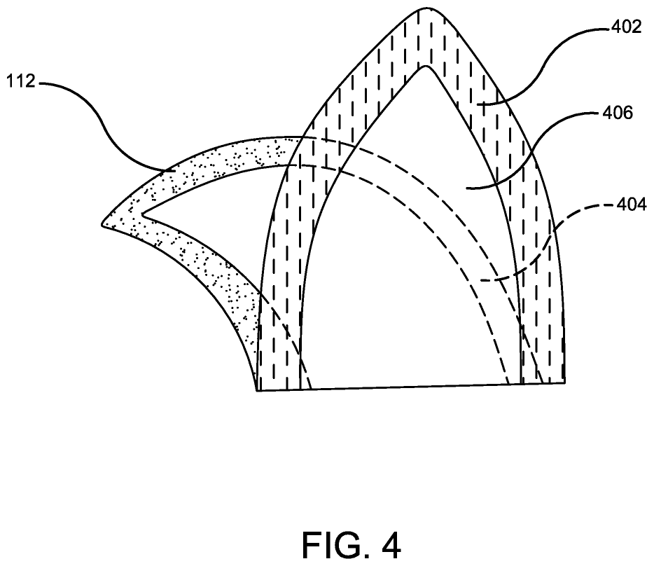

FIG. 4 illustrates a perspective view showing how the removable crotch area is removed from the compression garment device for restroom use. The removable crotch area 112 is configured to be removed, allowing a user to use a restroom without removing the entire compression garment device 100. The crotch area 112 is attached to the garment device 100 using hook and loop fasteners, or any other suitable fasteners as are known in the art. The hook and loop fastener 402 is positioned on the garment device 100, and the hook and loop fastener 404 is disposed on the crotch area 112. The hook and loop fastener 402 and the hook and loop fastener 404 fasten to each other (or mate) to attach the removable crotch area 112 to the garment device 100. When removing the removable crotch area 112, the hook and loop fastener 402 and the hook and loop fastener 404 are unfastened or pulled apart, to create an opening 406 for a user to use a restroom.

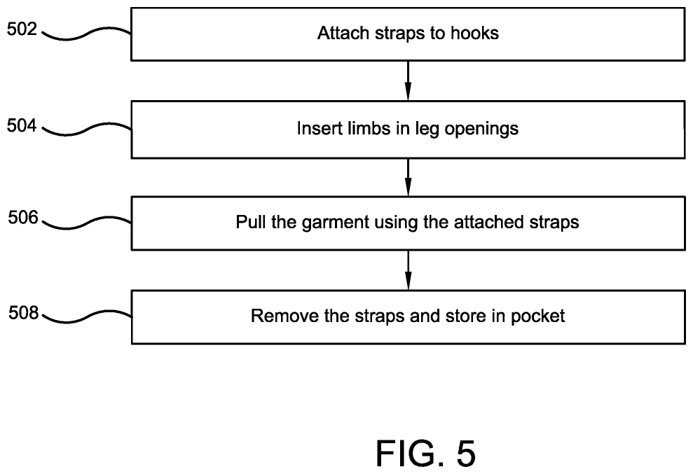

FIG. 5 illustrates a flow diagram showing the steps of pulling up the compression garment device over a user's body. Initially at 502, a plurality of straps is fastened to a plurality of hooks that are sewn to the compression garment device, wherein each strap is sewn to a pair of hooks. At 504, a user's limbs are inserted into the leg openings of the compression garment device. Then, at 506 a user easily pulls on the compression garment device by grasping the straps for a firm and secure grip. At 508, when the garment is in use, the straps are removed and stored in a pocket disposed on the garment device for a seamless appearance.

Consequently, by incorporating the straps and hooks in the compression garment device 100, the present invention facilitates the pulling on and off of the garment device 100. As described above, traditional compression garments require the user to pull up the garment from his or her limbs without a secure grip which can be difficult, especially for users who are injured or post-surgical or post-partum. In contrast, the compression garment device 100 utilizes the attached straps to allow a user to easily pull up the garment device 100. In addition, the user can simply remove the crotch portion from the garment device 100 when needing to use a restroom.

Certain terms are used throughout the following description and claims to refer to particular features or components. As one skilled in the art will appreciate, different persons may refer to the same feature or component by different names. This document does not intend to distinguish between components or features that differ in name but not structure or function. As used herein “improved compression garment device”, “compression garment device”, “garment device”, and “hooked compression garment device” are interchangeable and refer to the compression garment device 100 of the present invention.

Notwithstanding the forgoing, the compression garment device 100 of the present invention can be of any suitable size and configuration as is known in the art without affecting the overall concept of the invention, provided that it accomplishes the above-stated objectives. One of ordinary skill in the art will appreciate that the size, configuration, and material of the compression garment device 100 as shown in FIGS. 1-5 is for illustrative purposes only, and that many other sizes and shapes of the compression garment device 100 are well within the scope of the present disclosure. Although the dimensions of the compression garment device 100 are important design parameters for user convenience, the compression garment device 100 may be of any size that ensures optimal performance during use and/or that suits the user's needs and/or preferences.

Various modifications and additions can be made to the exemplary embodiments discussed without departing from the scope of the present invention. While the embodiments described above refer to particular features, the scope of this invention also includes embodiments having different combinations of features and embodiments that do not include all of the described features. Accordingly, the scope of the present invention is intended to embrace all such alternatives, modifications and variations as fall within the scope of the claims, together with all equivalents thereof.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. An improved compression garment device configured to be worn over a user's lower body, the improved compression garment device comprising:
a front surface which adheres to a front body portion of a user;
a rear surface which adheres to a rear body portion of the user;
a left leg opening;
a right leg opening;
a material section which extends up a torso of the user; and
a crotch area disposed to cover a crotch region of the user, wherein the improved compression garment device is a one-piece component.

2. The improved compression garment device of claim 1 further comprising a torso band wrapping around the torso of the user.

3. The improved compression garment device of claim 1, wherein the crotch area is removable to allow the user restroom access.

4. The improved compression garment device of claim 3, wherein the crotch area uses hook and loop fasteners to removably secure the crotch area to the improved compression garment device.

5. The improved compression garment device of claim 1 further comprising a plurality of removably secured straps positioned on the front surface and the rear surface.

6. The improved compression garment device of claim 5, wherein some of the plurality of removably secured straps are positioned in a vertical position on the front surface and the rear surface.

7. The improved compression garment device of claim 6, wherein some of the plurality of removably secured straps are positioned in a horizontal position on the front surface and the rear surface.

8. The improved compression garment device of claim 7 further comprising a pocket positioned on the front surface.

9. The improved compression garment device of claim 8, wherein the plurality of removably secured straps are removed and stored in the pocket.

10. The improved compression garment device of claim 9, wherein the plurality of removably secured straps each comprise a loop at each opposing end of a strap.

11. The improved compression garment device of claim 10, wherein a plurality of paired hooks are disposed on the front surface and the rear surface.

12. The improved compression garment device of claim 11, wherein the loop of the strap engages the plurality of paired hooks, securing the strap.

13. The improved compression garment device of claim 12, wherein the loop clips onto the plurality of paired hooks.

14. An improved compression garment device configured to be worn over a user's lower body, the improved compression garment device comprising:
a front surface which adheres to a front body portion of a user;
a rear surface which adheres to a rear body portion of the user;
a left leg opening;
a right leg opening;
a material section which extends up a torso of the user;
a torso band wrapping around the torso of the user;
a removable crotch area disposed to cover a crotch region of the user and to allow the user restroom access;
a plurality of removably secured straps positioned on the front surface and the rear surface, wherein the plurality of removably secured straps each comprise a loop at each opposing end of a strap; and
a plurality of paired hooks disposed on the front surface and the rear surface, wherein the improved compression garment device is a one-piece component, and further wherein the loop of the strap engages the plurality of paired hooks, securing the strap.

15. The improved compression garment device of claim 14 further comprising a pocket positioned on the front surface.

16. The improved compression garment device of claim 15, wherein the plurality of removably secured straps are removed and stored in the pocket.

17. The improved compression garment device of claim 14, wherein the removable crotch area uses hook and loop fasteners to removably secure the removable crotch area to the improved compression garment device.

18. The improved compression garment device of claim 14, wherein the plurality of removably secured straps are positioned in a horizontal or vertical position on the front surface and the rear surface.

19. A method of pulling up the improved compression garment device over a user's lower body, the method comprising the steps of:
fastening a plurality of straps to a plurality of hooks that are sewn to the improved compression garment device;
inserting a user's limbs into leg openings of the improved compression garment device;
grasping the plurality of straps to pull on the improved compression garment device;
once in use, removing the plurality of straps; and
storing the plurality of straps in a pocket disposed on the improved compression garment device for a seamless appearance.

20. The method of claim 19 further comprising a step of removing a crotch area disposed to cover a crotch region of a user, to allow the user restroom access
